(19) United States
(12) Patent Application Publication
(10) Pub. No.: US 2009/0124548 A1
(43) Pub. Date: May 14, 2009

(54) Method of Using Fish Plasma Components to Inhibit Glial Scarring and Promote Functional Recovery in the Mammalian CNS

(76) Inventor: Evelyn S. Sawyer, Freeport, ME (US)

Correspondence Address: IP STRATEGIES, 12 1/2 WALL STREET, SUITE E, ASHEVILLE, NC, 28801, US

(21) Appl. No.: 12/265,238

(22) Filed: Nov. 5, 2008

Related U.S. Application Data
(63) Continuation-in-part of application No. 11/223,791, filed on Sep. 8, 2005.
Application No. 11/223,791 is a continuation-in-part of application No. 11/019,083, filed on Dec. 21, 2004, now abandoned.
Application No. 11/019,083 is a continuation of application No. 10/418,189, filed on Apr. 17, 2003, now Pat. No. 6,861,255.
Application No. 10/418,189 is a continuation-in-part of application No. 09/907,443, filed on Jul. 18, 2001, now Pat. No. 6,599,740.
(60) Provisional application No. 60/255,451, filed on Dec. 15, 2000.

(51) Int. Cl. A61K 38/17; A61P 25/00

(52) U.S. Cl. 514/12

(57) ABSTRACT

A method includes applying salmon fibrin at a central nervous system injury site. For example, applying salmon fibrin can include injecting salmon fibrin. The method can also include causing the suppression of astrocyte activation, whereby glial scarring is at least reduced. The functional recovery of a patient who has suffered a central nervous system injury is promoted according to this method.

### CROSS-REFERENCE TO RELATED APPLICATION

This is a continuation-in-part of co-pending U.S. patent application Ser. No. 11/223,791, filed on Sep. 8, 2005, the entirety of which is incorporated herein by reference; which in turn is a continuation-in-part of U.S. patent application Ser. No. 11/019,083, filed on Dec. 21, 2004; which in turn is a continuation of U.S. patent application Ser. No. 10/418,189, filed on Apr. 17, 2003, now U.S. Pat. No. 6,861,255, which issued on Mar. 1, 2005; which in turn is a continuation-in-part of U.S. patent application Ser. No. 09/907,443, filed on Jul. 18, 2001, now U.S. Pat. No. 6,599,740, which issued on Jul. 29, 2003; which in turn is related to and claims priority from U.S. Provisional Patent Application No. 60/255,451, which was filed on Dec. 15, 2000. Priority is also claimed from U.S. Provisional Patent Application No. 60/986,747, which was filed on Nov. 9, 2007.

### FIELD OF THE INVENTION

This invention relates to methods of enhancing repair of the central nervous system by suppressing astrocyte activation that produces a glial scar.

### BACKGROUND OF THE INVENTION

Injury to the central nervous system (CNS), including the brain and spinal cord, often results in death or profound disability, exacting a heavy toll on individuals and society. Therefore, there is a great need for treatments that promote repair of damaged nerves and neural pathways.

Injury to the mammalian CNS results in a reaction leading to a glial scar. This reaction recruits and activates astrocytes and other cells, which produce inhibitors such as proteoglycans to form a molecular and physical barrier to regenerating axons (Silver et al. 2004), and hinder functional recovery (Fawcett et al. 2006). Therefore, axonal regrowth would be promoted by suppressing the astrocyte activation and recruitment that lead to the glial scar.

A number of methods, physical, pharmaceutical, and cellular, have been proposed or tested to suppress or overcome glial scarring. Transplantation of peripheral nerves to the CNS (Cheng et al. 1996), modification of substrate stiffness (Georges et al. 2006), physical channels made from peptide fibers (Ellis-Behnke et al. 2006), hydrogels (Tsai et al. 2006), and synthetic polymers are examples of conventional physical approaches. Suppression or blocking of inhibitory molecules by antibodies (Liebscher et al. 2005) or other molecules including Rho antagonists (Dergham et al. 2002), chondroitinase (Curing a et al. 2007), decorin Davies et al. 2006), tumor necrosis factor (Schwartz, 1996) and polysialic acid (El Maarouf et al. 2006) are examples of the pharmaceutical approach. Cellular approaches to overcome glial scarring include implantation of glial-restricted precursor cells (Rao et al. 2007), autologous macrophages (Eisenbach-Schwartz et al. 2001), and human embryonic stem cells (Kierstead et al. 2007).

Mammalian central nervous systems have a negligible capacity to regenerate after injury. This contrasts with the ability of lower vertebrates, including fish, to regenerate CNS tissue. However, fish plasma components are not conventionally used in vitro or in vivo and are actually discouraged for use in such situations, for several reasons, including:
1. Fish whole serum or plasma has failed to supplement or replace mammalian counterparts in other areas, such as in the media used for mammalian cell culture, due to the frequent toxicity and ineffectiveness of the fish material.
2. Fish are traditionally considered to be free-ranging, wild animals. Therefore, apparent uncertainty in quality, availability, and reproducibility of their blood products would seem to make them unsuitable donors.
3. The usual, and most cost-effective, method of fractionating human or other mammalian serum or plasma proteins (Cohn process) is not suitable for salmon or other coldwater fish, since the separation depends in part on temperature effects. Since salmon plasma can vary in temperature from 0° C. to 16° C. seasonally, this method is unreliable.
4. Fish plasma proteins have been studied from the perspective of comparative physiology and evolution, and found only partially identical to their mammalian homologues (Doolittle, 1987). For example, salmon transferrin has only a 40-44% amino acid sequence identity with human transferrin (Denovan-Wright et al., 1996). This and similar data for other plasma proteins such as fish albumin (Davidson et al., 1989) would dissuade those skilled in the field from trying other fish plasma components.
5. Compared to plasma from mammals, salmon and trout plasma contain oxidative enzymes that remain active at low temperatures, and therefore are likely to generate cytotoxic substances. Therefore, special preparation and handling procedures are required.
6. To the mammalian immune system fish plasma proteins are foreign proteins and likely to elicit an antibody response. Laidmae et al. (2006) showed that salmon fibrinogen and thrombin did indeed produce antibodies in host animals. However, these antibodies did not react with the host's protein.

### BRIEF SUMMARY OF THE INVENTION

Nonetheless, no safe, effective method of overcoming glial scarring and permitting neuron regrowth in the mammalian CNS has been developed. It is an object of this invention to demonstrate that salmon fibrinogen and thrombin, that is, salmon fibrin, injected at the injury site can promote suppression of astrocyte activation and therefore glial scarring, resulting in measurable and significant functional recovery.

According to an aspect of the invention, a method includes applying salmon fibrin at a central nervous system injury site. For example, applying salmon fibrin can include injecting salmon fibrin.

The method can also include causing the suppression of astrocyte activation, whereby glial scarring is at least reduced.

According to another aspect of the invention, the functional recovery of a patient who has suffered a central nervous system injury is promoted according to the method described above.

The method can also include obtaining a salmonid that is a progeny of domesticated broodstock that are reared under consistent and reproducible conditions. Blood is obtained from the fish, plasma is separated from the blood, and the salmon fibrin is extracted from the plasma. Preferably, the salmonid from which the blood is obtained is sexually immature, in the log-phase of growth, larger than two kilograms, and/or reared by standard husbandry methods. Obtaining blood from the salmonid can include rendering the salmonid to a level of loss of reflex activity, and drawing blood from a caudal blood vessel. Prior to rendering the salmonid to a level of loss of reflex activity, the levels of proteolytic enzymes and non-protein nitrogen present in the blood of the salmonid are preferably reduced. Separating plasma from the blood can include centrifuging the blood. Extracting the salmon fibrin from the plasma can include performing an extraction process on the plasma such that all process temperatures are no greater than 4° C., no cytotoxic chemical residues remain in the one or more plasma components, and no oxidation of plasma lipids occurs.

The method can also include adding an antioxidant and/or a protease inhibitor to the plasma prior to extracting the salmon fibrin.

Preferably, the salmonid is an Atlantic salmon.

### BRIEF DESCRIPTION OF THE DRAWING FIGURES

FIG. 1 illustrates the resistance to changes in pH and osmolality of salmon fibrin gel.

FIG. 2 shows mammalian neurons grown in bovine fibrin gels.

FIG. 3 shows mammalian neurons grown in fish fibrin gels.

FIG. 4 is a graph depicting the difference in average total neurite length per cell of mammalian neurons grown in bovine fibrin gels and mammalian neurons grown in fish fibrin gels.

FIG. 5 is a spinal cord injury-BBB graph.

FIG. 6 is a graph showing the average volume of urine manually expressed from bladders of rats treated with salmon or human fibrin after a T9 hemisection injury.

FIG. 7 shows images of spinal cords of animals treated with salmon fibrin 2 days post-surgery, stained with antibodies.

FIG. 8 shows images of spinal cords of animals treated with salmon fibrin 2 days post-surgery, stained with antibodies.

FIG. 9 shows images of spinal cords of animals treated with salmon fibrin 5 days post-surgery, stained with antibodies.

FIG. 10 shows images of sections of spinal cords of injured rats, stained with a GFAP antibody to detect glial scar formation.

FIG. 11 is a chart showing the degree of glial scar formation in injured spinal cords.

### DETAILED DESCRIPTION OF THE INVENTION

According to the invention, salmon fibrin is applied, preferably by injection, at the CNS injury site to promote suppression of astrocyte activation and therefore glial scarring, resulting in measurable and significant functional recovery.

Because of the many risks and uncertainties inherent in human and other mammalian biologics, and the cytotoxicity and ineffectiveness of fish whole serum or plasma, it is preferred that fish plasma components used in connection with the method of the present invention are separated (purified) from the whole plasma of farmed fish. Fish species for which consistent and reproducible methods of production are well established are best suited for use in the method of the present invention. Exemplary use of salmonids, specifically the Atlantic salmon (Salmo salar), will be described and demonstrated; however, the scope of the present invention is not limited to use of this particular species.

According to the method of the present invention, advantages of the use of fish plasma components are exploited. The method of the present invention takes advantage of the fact that commercial salmon aquaculture has grown dramatically in recent years. In Maine alone, there are over six million fish, averaging 4-6 kilograms each, reared in offshore pens annually. The availability of raw material (blood) and the efficiency of recently developed blood-drawing methods and devices contribute to a large supply and availability of fish blood. By utilizing these domesticated fish stocks reared in aquaculture facilities, plasma can be obtained with product consistency similar to plasma from, for example, herds of cattle reared for this purpose.

Further, although amino acid sequences in fish and mammalian plasma proteins have less than 50% identity, many of the critical sequences or active sites required for similar function in both fish and mammals, are highly-conserved among vertebrates including salmon and trout.

Salmonid plasma components are unlikely to transmit mammalian infections agents. The wide evolutionary distance between fish and mammals, and the differences in body temperature between mammals and the cold-water fishes such as trout and salmon, provide safety from cross-species infection.

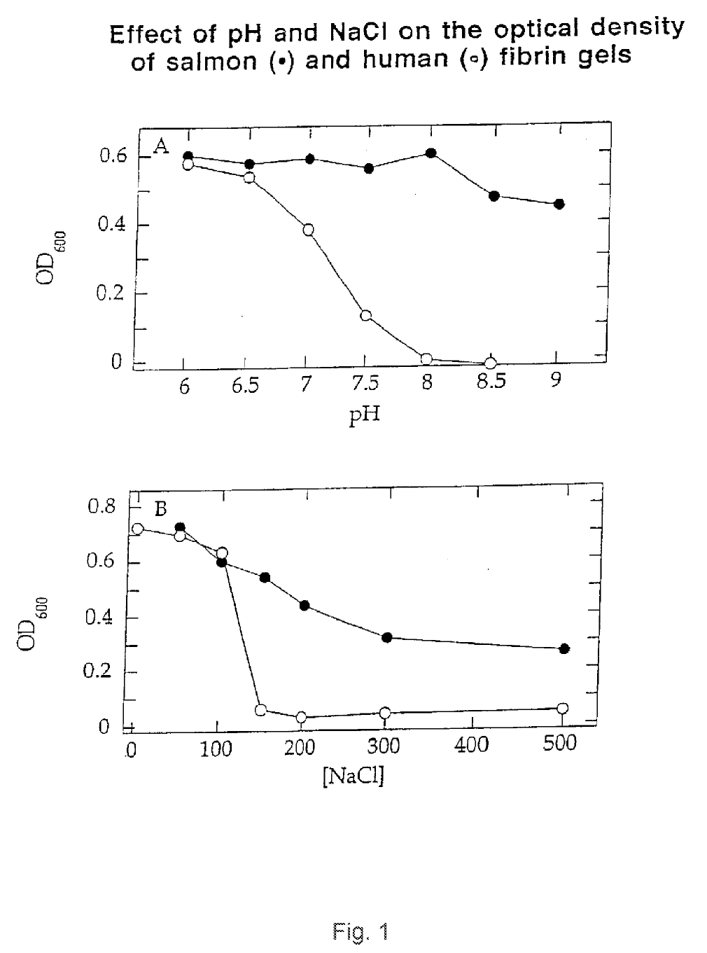

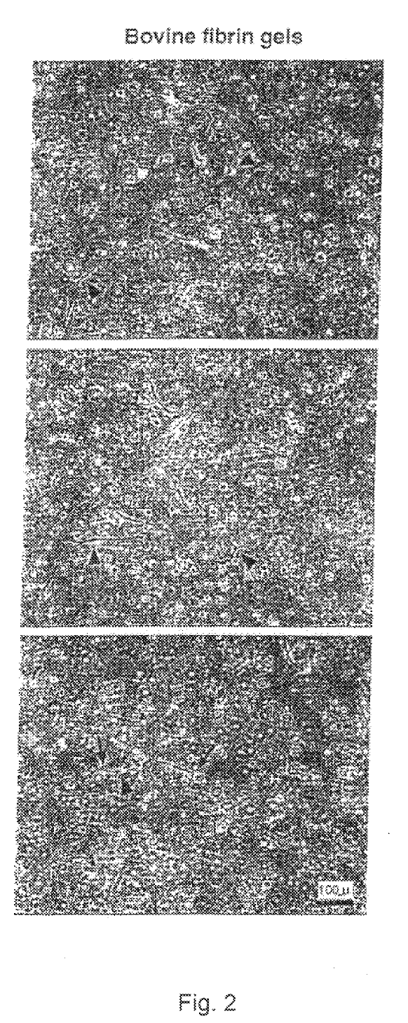

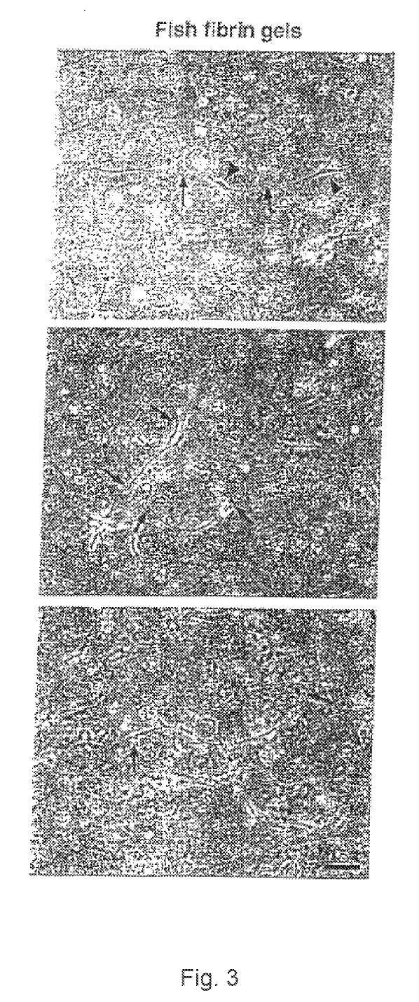

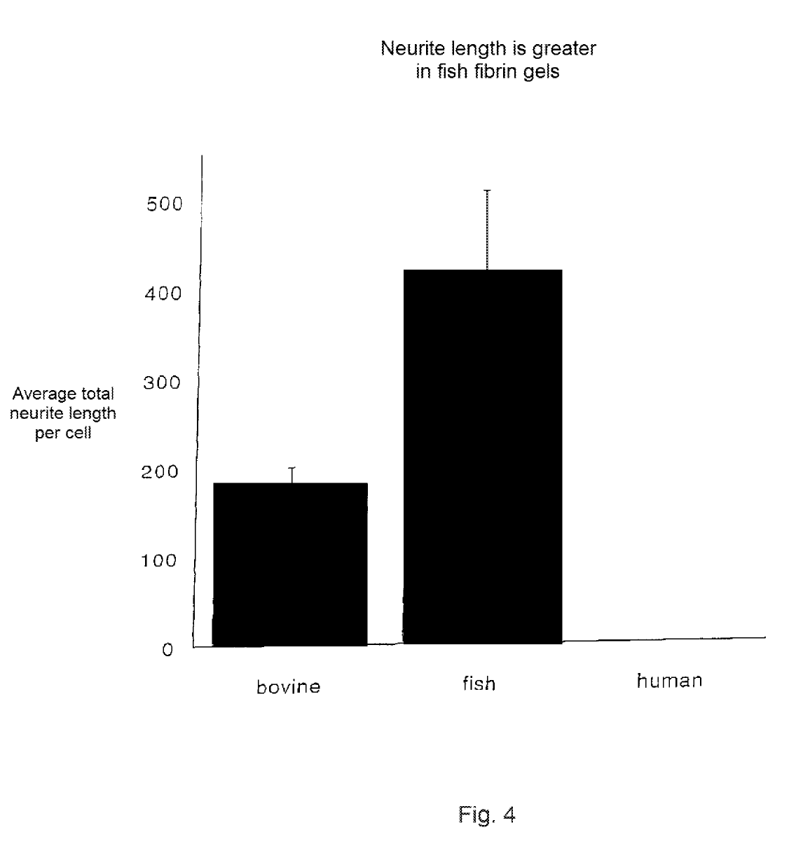

Salmonid plasma components are more effective than mammalian products for certain applications. Because salmon lipids and plasma proteins must function in vivo over a wide range of temperature, pH, and osmolality, their performance in tissue culture reflects these properties. Salmon lipids are highly unsaturated and rich in omega-3 fatty acids. Lypholized salmon fibrinogen is easily reconstituted at room temperature, unlike lyophilized mammalian fibrinogens, which must be heated to 37° C. (Catalog 1999, Calbiochem, San Diego, Calif.). Gels produced with salmon fibrinogen and thrombin are more resistant to changes in pH and NaCl concentration than gels made with human proteins (FIG. 1). Mammalian neurons grown in salmon gels show enhanced process outgrowths compared to neurons grown in mammalian gels (FIGS. 2-4).

Salmonid plasma components can be produced with lot-to-lot consistency. An important requirement is for donor fish to be reared under consistent and reproducible conditions, not necessarily the nature or specifics of these conditions. The reproducibility of conditions reduces variability in quantity and quality of plasma components.

The physiology of fishes, including plasma composition, is regulated to a much greater degree by external factors than that of mammals. Therefore, plasma composition can be manipulated by environmental or nutritional means not possible in mammals. For example, amounts of cholesterol and high-density lipoprotein (HDL) are significantly different in salmon held at different salinities or fed different diets. (Babin and Vernier, 1989).

According to the present invention, fibrinogen and thrombin from the plasma of Atlantic salmon (S. salar) was used for the disclosed examples because consistent and reproducible methods for their production are well established, large numbers are reared in commercial aquaculture operations, and individual fish are large enough for blood to be obtained easily.

The process begins with the consistent and reproducible conditions under which donor fish are reared. All fish used as plasma sources preferably are progeny of domesticated broodstock, inspected for fish disease according to the American Fisheries Society “Blue Book” standards, sexually immature, in the log-phase of growth, larger than two kilograms, reared by standard husbandry methods, and fed a commercially pelleted food appropriate to the species.

Water temperature at the time of bleeding is preferably 4° C. to 12° C. The fish are preferably starved for five days before bleeding to reduce proteolytic enzymes and non-protein nitrogen. Each fish is stunned, such as by a blow to the head, or by immersion in ice-water, or in water containing CO₂ or other fish anesthetic, in order to render the fish to a level of loss of reflex activity (unconsciousness) as defined by Schreck and Moyle, (1990). Whole blood is then drawn, preferably from the caudal artery or vein with a sterile needle and a syringe or vacuum tube containing an anticoagulant such as ACD (acid citrate dextrose), trisodium citrate, or other anticoagulant commonly used in human blood-banking.

Whole blood is held for no more than four hours at 2°-4° C., and then centrifuged at 2°-4° C. Because of the large amounts of highly unsaturated fatty acids, plasma to be used for lipid extraction preferably is handled under argon, or an antioxidant such as alphatocopherol, BHT, or mercaptoethanol at less than 1 ppm is added. Plasma is then frozen, for example, at −80° C.

For fibrinogen extraction and purification, the method of Silver et al., 1995 preferably is used. This method is based on ammonium sulfate precipitations, which yields greater than 95% pure fibrinogen (by SDS-PAGE). Preferably, thrombin is prepared by the method of Ngai and Chang, 1991.

These extraction techniques are illustrative of those currently in use, but other techniques may be equally effective. The essential requirements are that all process temperatures must remain below 4° C. and there must be no cytotoxic chemical residues in the product.

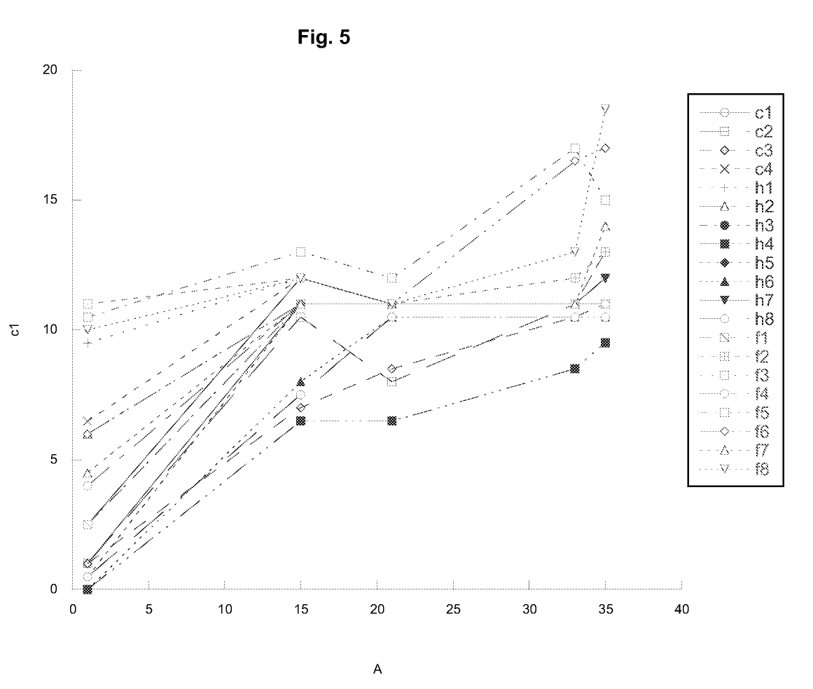

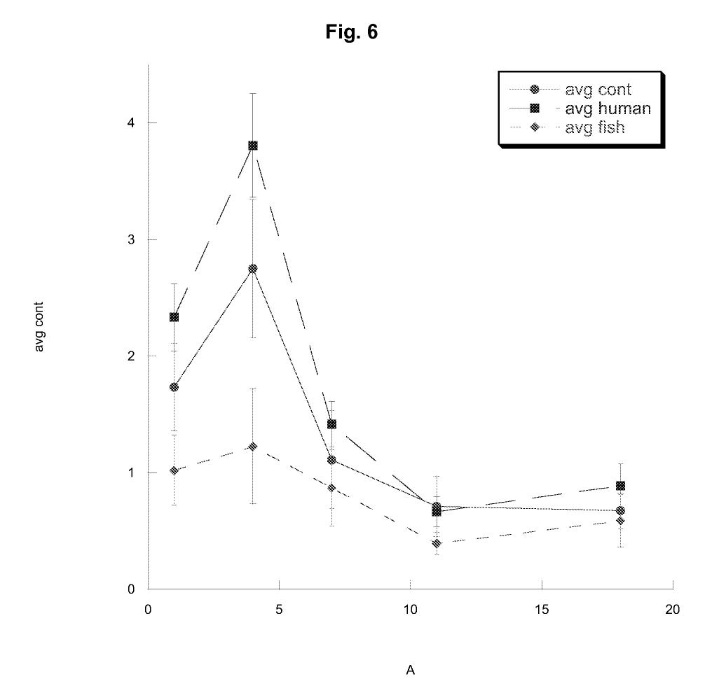

FIG. 5 shows the Basso, Beattie, and Bresnahan (BBB) scores of rats, treated with salmon (N=8) or human fibrin (N=8) or untreated controls (N=8) after spinal cord injuries (T9 hemisection injury). The day post-surgery is shown on the x-axis and the BBB score is shown on the y-axis. Error bars represent standard error of the mean. The beneficial effects of the salmon fibrin at the earliest timepoint suggest hemostasis and sparing of axonal fibers. This graph shows significant functional recovery in animals treated with the salmon fibrin. This result is further supported by FIG. 6, which shows the average volume of urine manually expressed from bladders of the rats treated with salmon or human fibrin after the T9 hemisection injury. A lower volume of urine correlates with better bladder function.

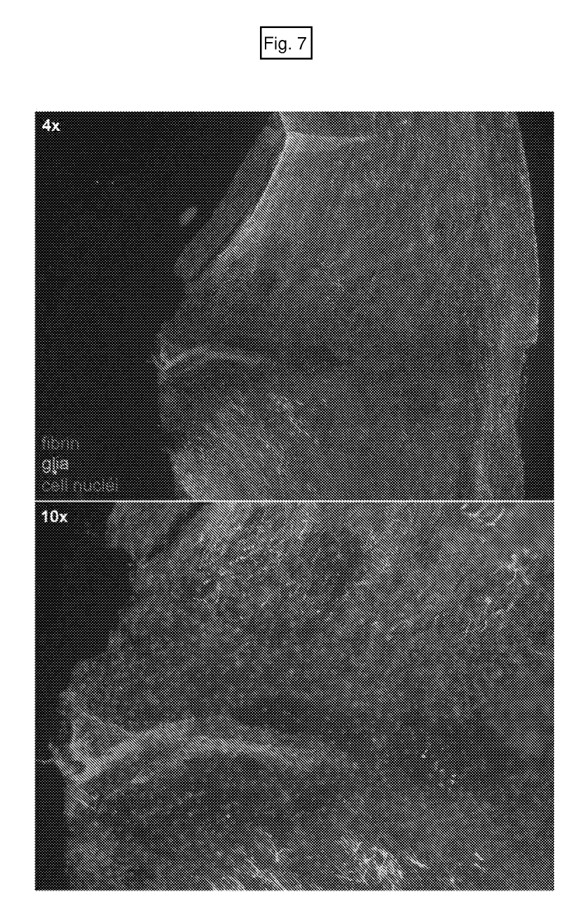

FIG. 7 shows an image of spinal cords of animals treated with salmon fibrin two days post-surgery, stained with antibodies to salmon fibrin (red), glia (astrocytes) for GFAP (Glial fibrillary acidic protein) (green), and cell nucli stained with Hoechst (blue). As shown, reactive astrocytes are confined to the periphery of the lesion and do not infiltrate the fibrin gel.

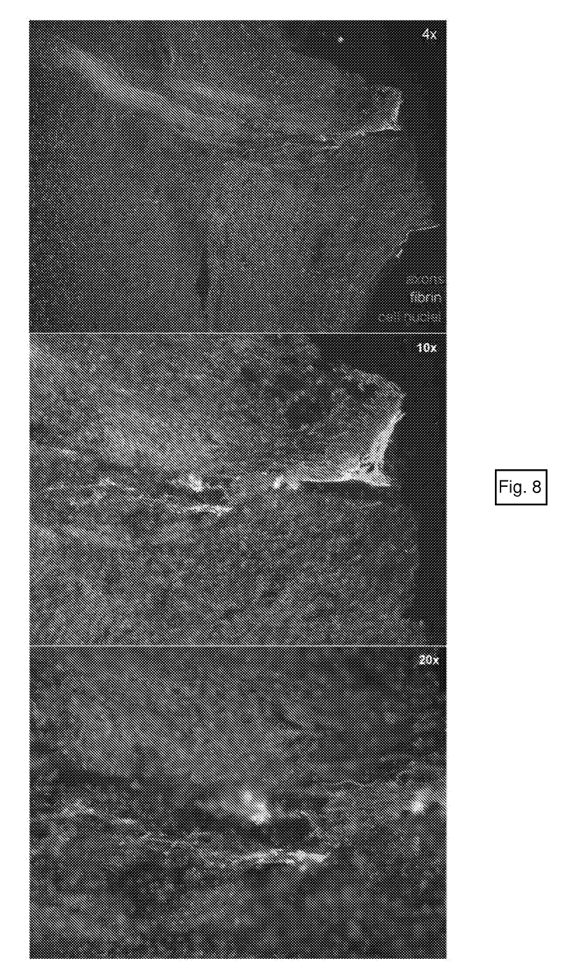

FIG. 8 shows an image of spinal cords of animals treated with salmon fibrin 2 days post-surgery, stained with antibodies to salmon fibrin (green), axons with neurofilament-H (an axon marker) (red) and cell nuclei with Hoechst (blue). As shown, axons surround the salmon fibrin at the lesion site, a sign of CNS recovery.

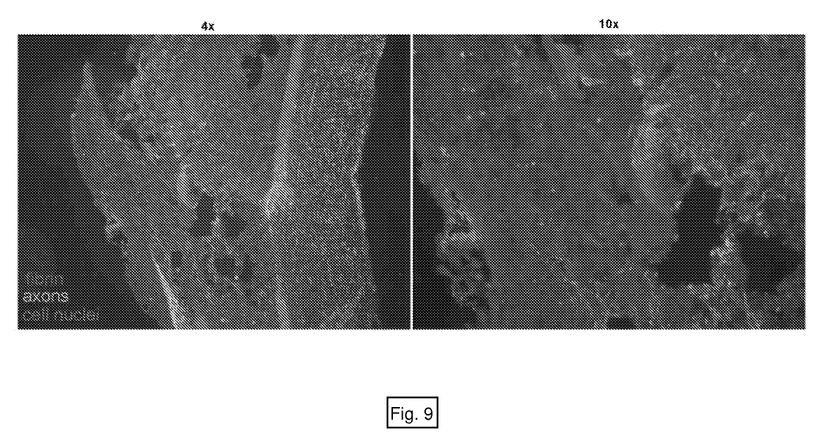

FIG. 9 shows an image of spinal cords of animals treated with salmon fibrin 5 days post-surgery, stained with antibodies to salmon fibrin (red), axons with neurofilament-H (green), and cell nuclei with Hoechst (blue). As shown, axons surround the salmon fibrin at the lesion site, a sign of CNS recovery.

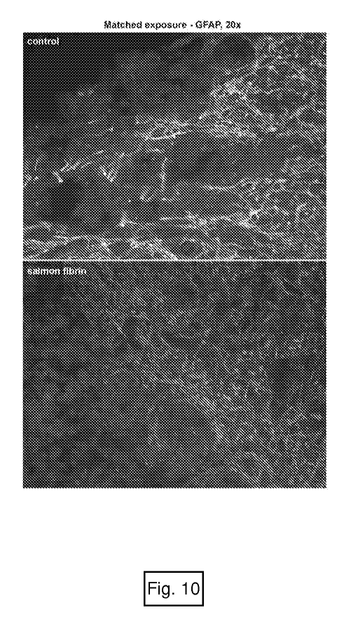

FIG. 10 shows an image of sections of spinal cords of injured rats, stained with a GFAP antibody to detect glial scar formation. A greater degree of glial scar is evident in sections from control animals compared to animals treated with salmon fibrin (the images are matched exposures).

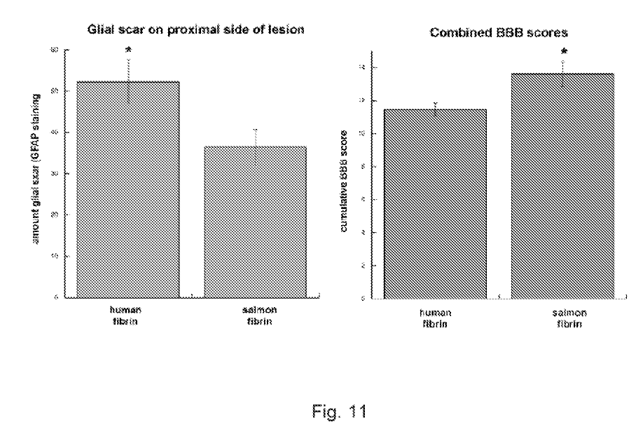

As shown in FIG. 11, the degree of glial scar formation in injured spinal cords was analyzed by measuring the amount of GFAP staining in ˜0.04 mm² regions along the lesion. N=8 animals per condition; staining intensity was normalized to unlesioned areas for each section. As shown, application of salmon fibrin produces less glial scarring and better functional recovery than use of human fibrin.

### EXAMPLE

Adult rats were anesthetized, subjected to a dorsal hemisection spinal cord lesion (Grill et al. 1997), and treated with human and salmon fibrin gels of equal stiffness (Georges et al. 2006). The animals were injected at the lesion site with either 3 mg/ml salmon fibrin or 3 mg/ml human fibrin (Tisseal), or received no treatment. Both the salmon fibrin and the human fibrin were applied by simultaneous injection of 3 mg/ml fibrinogen and 1.5 units thrombin. After treatment, the animals were sutured and allowed to recover from surgery. The animals were not treated with immunosuppressive drugs, and received manual bladder care post-surgery until bladder function recovered. Function post-surgery as defined by locomotor behavior was assessed by BBB testing (Basso et al 1996) beginning one day after surgery and continuing until ˜11 weeks post-surgery. Sensory function was assessed ˜10 weeks post-surgery.

### Summary of Results:

BBB testing—The animals treated with salmon fibrin performed better than those treated with human fibrin or untreated controls. Repeated measures of analysis of variance (ANOVA) statistical analysis shows that the salmon fibrin group was significantly different from the control group (p<0.05) while the human fibrin group was not (p=0.276). The BBB results also suggest that the beneficial effect of the salmon fibrin occurred early, since the salmon fibrin group was significantly different from controls at the earliest time point (1 day after treatment, p<0.01). (FIG. 5)

Bladder function—Animals treated with salmon fibrin recovered bladder function more rapidly than those treated with human fibrin or untreated controls. (FIG. 6)

Sensory testing—There was no obvious difference in the sensory response of animals in any of the treatment groups.

Immunohistochemistry—Staining cryosections of spinal cords with an antibody to salmon fibrin showed that intact fibrin gel is present in the lesion site 2 to 5 days after treatment. The anti-salmon fibrinogen antibody does not recognize endogenous rat fibrin.

The lesion site containing salmon fibrin was surrounded by axons (FIGS. 8 and 9) rather than activated astrocytes. These astrocytes, which produce glial scarring, were confined to the periphery of the lesion (FIG. 7). Comparison of GFAP staining, indicative of glial scarring, shows greater intensity in control vs. salmon fibrin treated animals (FIG. 10)

### Conclusion:

The exemplary experiment demonstrates that salmon fibrin suppresses astrocyte activation and therefore the glial scar, resulting in significantly enhanced functional recovery.

### REFERENCES

U.S. Pat. Nos.:
5,580,555 Schwartz M. Dec. 3, 1996.
6,267,955 Eisenbach-Schwartz M, Speigler O, Hirshberg D L. Jul. 31, 2001.
7,214,372 Rao M S, Noble M, Mayer-Proschel M. May 8, 2007
7,285,415 Keirstead H S, Nistor G I. Oct. 23, 2007.

### Publications:

Babin, P. J. and Vernier, J. 1989. Plasma Lipoproteins in Fish. J. Lipid Research 30:467-489.
Basso D M, Beattie M S, Bresnahan J C. 1995. A sensitive and reliable locomotor rating scale for open field testing in rats. J. Neurotrauma 12:1-21.
Cheng H, Cao Y. Olson L. 1996. Spinal cord repair in adult paraplegic rats: partial restoration of hind limb function. Science 273:510-13.
Curing a G M, Snow D M, Mashburn C, Kohler K, Thobaben R, Caggiano A O, Smith G M. 2007. Mammalian-produced chondroitinase AC mitigates axon inhibition by chondroitin sulfate proteoglycans. J. Neurochem 102(1):275-88.
Davies J E, Tang X, Bournat J C, Davies S J. 2006. Decorin promotes plasminogen/plasmin expression within acute spinal cord injuries and by adult microglia in vitro. J. Neurotrauma 23(3-4):397-408.
Dergham P, Ellezam B, Essagian C, Avedissian H, Lubell W D, Mckerracher L. 2002 Rho signaling pathway targeted to promote spinal cord repair. J. Neuroscience 22(15):6570-77.
Ellis-Behnke R G, Liang Y X, You S W, Tay D K C, Zhang S, So K F, Schneider G E. 2006. Nano neuro-knitting: Peptide nonofiber scaffold for brain repair and axon regeneration with functional return of vision. Proc Natl Acad Sci 103(13):5054-59.
El Maarouf A, Petridis A K, Rutishauser U. 2006. Use of polysialic acid in repair of the central nervous system. Proc. Natl. Acad. Sci. 103(45):16989-94.
Fawcett J W, Asher R A. 1999. The glial scar and central nervous system repair. Brain Res, Bull. 49(6):377-91.
Georges P C, Miller W J, Meaney D F, Sawyer E S, Janmey P A. 2006. Matrices with compliance comparable to brain tissue select neuronal over glial growth in mixed cortical cultures. Biophysical J. 90(8):3012-18.
Grill R, Murai K, Blesch A, Gage F H, Tuszynski M H. 1997. Cellular delivery of neurotrophin-3 promotes cortocospinal axonal growth and partial functional recovery after spinal cord surgery. J. Neuroscience 17(14): 5560-72.
Liebscher T, Schnell L, Schnell D, Scholl J, Schneider R, Gullo M, Fouad K, Mir A, Rausch M, Kindler D, Hammers F P, Schwab M E. 2005. Nogo-A antibody improves regeneration and locomotion of spinal cord injured rats. Ann. Neurol. 58(5):706-19.
Laidmae I, McCormick M E, Herod J H, Pastore J J, Sawyer E S, Janmey P A, Uibo R. 2006. Stability, sterility, coagulation, and immunologic studies of salmon coagulation proteins with potential use for mammalian wound healing and cell engineering. Biomaterials 27(34):5771-9.
Ngai, P. K., and Chang, J. Y., 1991. A Novel One-Step Purification of Human Thrombin After Direct Activation of Crude Prothrombin Enriched From Plasma. Biochem J. 280:805-808.
Schreck, C. B. and Moyle, P. B. 1990. Methods for Fish Biology, Am. Fish. Soc. Bethesda, Md.
Silver, F. R., Wang, M. C., and Pins, G. D. 1995. Preparation of Fibrin Glue: A Study of Chemical and Physical Methods. J. Appl. Biomaterials 6(3):175-183.
Silver J, Miller J H, 2004. Regeneration beyond the glial scar. Nature Neuroscience 5(2): 146-56.

Tsai E C, Dalton P D, Shoichet M S, Tator C H. 2006. Matrix inclusion within synthetic hydrogels guidance channels improves specific supraspinal and local axonal regeneration after complete spinal cord transection. Biomaterials 27(3):519-33.

1. A method, comprising applying salmon fibrin at a central nervous system injury site.

2. The method of claim 1, wherein applying salmon fibrin includes injecting salmon fibrin.

3. The method of claim 1, further comprising causing the suppression of astrocyte activation, whereby glial scarring is at least reduced.

4. A method of promoting the functional recovery of a patient who has suffered a central nervous system injury, including the method of claim 1.

5. The method of claim 1, further comprising:
obtaining a salmonid that is a progeny of domesticated broodstock that are reared under consistent and reproducible conditions;
obtaining blood from the fish;
separating plasma from the blood; and
extracting the salmon fibrin from the plasma.

6. The method of claim 5, wherein the salmonid from which the blood is obtained is at least one of sexually immature, in the log-phase of growth, larger than two kilograms, and reared by standard husbandry methods.

7. The method of claim 5, wherein obtaining blood from the salmonid includes:
rendering the salmonid to a level of loss of reflex activity; and
drawing blood from a caudal blood vessel.

8. The method of claim 7, wherein obtaining blood from the salmonid includes, prior to rendering the salmonid to a level of loss of reflex activity, reducing the levels of proteolytic enzymes and non-protein nitrogen present in the blood of the salmonid.

9. The method of claim 5, wherein separating plasma from the blood includes centrifuging the blood.

10. The method of claim 5, wherein extracting the salmon fibrin from the plasma includes performing an extraction process on the plasma such that:
all process temperatures are no greater than 4° C.;
no cytotoxic chemical residues remain in the one or more plasma components; and
no oxidation of plasma lipids occurs.

11. The method of claim 5, further comprising adding at least one of an antioxidant and a protease inhibitor to the plasma prior to extracting the salmon fibrin.

12. The method of claim 5, wherein the salmonid is an Atlantic salmon.
